(19) United States
(12) Patent Application Publication
(10) Pub. No.: US 2007/0098785 A1
(43) Pub. Date: May 3, 2007

(54) MEDICANT DELIVERY SYSTEM AND DEVICE

(76) Inventors: Tim Clarot, Phoenix, AZ (US); Regina Miskewitz, Phoenix, AZ (US)

Correspondence Address: SNELL & WILMER L.L.P. (Main), 400 EAST VAN BUREN, ONE ARIZONA CENTER, PHOENIX, AZ, 85004-2202, US

(21) Appl. No.: 11/163,876

(22) Filed: Nov. 2, 2005

(51) Int. Cl. A61K 9/66

(52) U.S. Cl. 424/455

(57) ABSTRACT

A medicant delivery system and device containing a pre-measured, multi-symptom relieving medicine dose is provided. In particular, the delivery system relates to highly viscous cold and/or flu relieving medicants conveniently packaged in single doses. The medicant contains a plurality of therapeutic ingredients and is flavor neutral. The viscosity of the medicant is high enough so that the medicant remains contained in the delivery portion with little or no spillage, but is low enough so that it can be dissolved into a variety of beverages at any temperature.

### FIELD OF INVENTION

The present invention relates to a medicant delivery device containing a pre-measured, multi-symptom relieving medicine dose. In particular, this invention relates to highly viscous cold and/or flu relieving medicants conveniently packaged in single doses to be mixed with any beverage at any temperature or taken orally from the delivery device.

### BACKGROUND OF INVENTION

Multi-symptom relieving medicants of various formulations are known in the art. In most instances, particularly as it relates to cold and flu symptoms and medicants for the relief thereof, the medicants are offered as pills, liquids and the like. Such dosage methodologies are common place, and as such, will not be described in detail here. However, it is noted that such medicant delivery methodologies often suffer from several problems. For example, pills may be difficult to swallow, whereas liquids often can be messy or difficult to properly measure appropriate dosages. Moreover, many liquids have unappealing tastes if taken orally and, if used with a beverage, interfere with the taste of the drink. Similarly, many medicant systems provide limited dosing options, e.g., usable only in hot water.

Accordingly, there is a need for a more convenient, effective medicant delivery system and device with multiple dosing options.

### SUMMARY OF INVENTION

The present invention is directed towards a medicant delivery device that provides a convenient, effective medicant delivery system and device. While the way in which the present invention does so will be discussed in greater detail herein, in general, the present invention provides a medicant provided in conjunction with a single dose delivery device which is configured and intended for mixing the medicant with other beverages at any temperature, or which may be taken orally from the delivery device itself.

In accordance with one embodiment of the present invention, the system comprises a device having a delivery portion and a handle portion and a medicant suitably contained within the delivery portion. The handle portion may be attached to the delivery portion, and as such, may advantageously enable the device to be dipped and stirred into a beverage, or to be taken orally. Preferably, the delivery portion forms a measured dosage cavity and a highly viscous multi-symptom medicant contained in the cavity. In accordance with a preferred embodiment of the present invention, viscosity of the medicant is on the order of about 6,000 to about 20,000 centipoise.

In accordance with another embodiment of the present invention, the medicant comprises the plurality of active ingredients selected from the group a cough suppressant, a decongestant, an antihistamine, and an analgesic, a thickener, and a pH adjustment solution and optional ingredients. Preferably, the medicant is dissolvable in any drinkable liquid at any temperature.

### DETAILED DESCRIPTION

The following descriptions are of exemplary embodiments of the invention only, and are not intended to limit the scope, applicability or configuration of the invention in any way. Rather, the following description is intended to provide convenient illustrations for implementing various embodiments of the invention. As will become apparent, various changes may be made in the function and arrangement of the elements described in these embodiments without departing from the spirit and scope of the invention.

In accordance with an embodiment of the present invention, a medicant delivery system and device comprises a delivery portion, a handle portion and a liquid medicant providing, for example, convenient storage, shipment and oral administration of medicant. The convenience of the delivery system may be achieved by packaging medicant in a single dose delivery device obviating the need for a consumer to properly measure appropriate dosages. Furthermore, the system removes the need to purchase and store separate medicant containers and delivery devices. For example, the delivery system would allow one to carry one or two pre-measured dose devices to work or school with less worry of spillage, incorrect measurements, or missing a dose. In this regard, the delivery system comprises any suitable configurations or components that enable convenient administration of pre-measured doses of medicant.

In accordance with various exemplary embodiments of the present invention, the delivery portion has a shape suitable for storing, shipping, holding and/or dispensing liquid substances. For example, in an exemplary embodiment, the delivery portion has a shape suitable for dipping into beverage containers and/or comfortably inserting into the mouth. In another embodiment, the delivery portion has a shape of a straw or dinner utensil. In a preferred embodiment, the delivery portion has a shape of the bowl portion of a serving spoon. However, any shape which enables medicant to flow out of the delivery portion while stirring or when taken orally, including being able to lick any remaining medicant from the delivery portion, is suitable.

As noted above, exemplary embodiments of the present invention comprise a handle portion. Generally, the handle portion is configured and attached to the delivery portion to enable manipulation of the delivery portion. In this regard, the handle portion can be used for stirring, dipping, or otherwise handling the delivery portion so that the medicant can remain untouched and contained until ready for administration. In accordance with various embodiments of the present invention, the handle portion may be removably attached from the delivery portion and may be disposed of or reused by reattaching to another delivery portion. In accordance with various non-limiting embodiments, the handle portion has the shape of a round stick or flattened shaft. Preferably, when the handle portion is attached to the delivery portion, the delivery device resembles a utensil such as a spoon. Of course, one skilled in the art will appreciate configurations are not so limited and numerous other “utensil-like” configurations such as measuring cups, or ladles may likewise be substituted.

In accordance with another aspect of the present invention, the medicant is contained in the delivery portion while delivery device is stored or in transit. In accordance with an embodiment of the invention, one or more coverings may be removably attached to, or surround the delivery portion and/or handle portion of the delivery device. In general, any covering which contains the medicant during storage and transit, and protects medicant from contamination is suitable. For example, one covering may be removably attached to the delivery portion, while a second covering may contain both the delivery and handle portions. In accordance with various non-limiting embodiments, the covering may be made of any material that is capable of adhering to the material of which the delivery or handle portion is made. Preferably, for example, the covering is made of foil or polylaminate.

In accordance with an exemplary embodiment, the covering can be removably attached to the delivery and/or handle portions using various adhesive techniques, but is preferably attached using heat or magnetic induction. Preferably, in accordance with various embodiments, when the covering is removed, little or no spillage of the medicant occurs.

As noted above, in various embodiments, medicant is contained in the delivery portion of medicant delivery system. Preferably, in such embodiments, the covering and delivery portion form a cavity appropriately formed and/or sized to contain one dose of medicant. In its various configurations, the cavity formed is preferably sized to contain one dose of medicant, though the sizing of the cavity need not necessarily be precise.

In accordance with an embodiment of the present invention, the medicant is a multi-symptom relieving liquid comprised of a plurality of active ingredients, a thickener, a pH adjustment solution, and optional ingredients such as a color solution. The viscosity of the medicant is high enough so that the medicant remains contained in the delivery portion while peeling off the seal with little or no spillage, but is low enough so that it can be dissolved into a variety of beverages at any temperature. For example, in one embodiment, the viscosity is between about 6,000 and 20,000 centipoise. Preferably, the viscosity is between about 8,000 and about 12,000 centipoise. While a more preferred viscosity is about 9,000 to about 11,000 centipoise.

In accordance with various embodiments of the invention, a medicant is selected that contains one or more active ingredients. For example, in a preferred embodiment, the active ingredients are chosen to relieve one or more symptoms of the cold or flu. Although various non-limiting embodiments are described below, any liquid that performs a therapeutic or medicinal function is suitable for the delivery system.

For example, in various exemplary embodiments, the active ingredients are selected from the group of a cough suppressant, a decongestant, an antihistamine, and an analgesic. Depending on the desired therapeutic effect, the number of active ingredients included in the composition, and the amount in the solution can be varied. For example, in one embodiment of the present invention, a formulation may be designed for relief of headache pain only. In this instance, only one active ingredient, such as an analgesic, will be selected. In contrast, in an alternative exemplary embodiment of the invention, a formulation designed for night or day time relief of headache, congestion, runny nose, cough and sore throat may contain a selection from all the active ingredients.

In accordance with an embodiment of the present invention, medicant comprises a formulation that suppresses or controls coughs. For example, in an exemplary embodiment, dextromethorphan hydrogen bromide is provided, preferably, in an amount from about 0.3 to about 0.5 percent.

In accordance with another embodiment of the present invention, medicant comprises a formulation that treats symptoms of the respiratory system, such as a decongestant. In an exemplary embodiment, the decongestant may be phenylephrine hydrochloride, preferably, in an amount from about 0.1 to about 0.15 percent.

In accordance with another embodiment of the present invention, medicant comprises a formulation that prevents or alleviates the major symptoms of an allergic response, for example, an antihistamine. In accordance with a preferred embodiment of the present invention, the antihistamine is present in an amount from about 0.03 to about 0.18 percent. In various embodiments of the present invention, the antihistamine may be configured as a night time or day time formulation as desired. For example, an exemplary embodiment of a night time formulation contains doxylamine succinate, preferably, present from about 0.13 to about 0.18 percent. Alternatively, an exemplary embodiment of a day time formulation contains chlorpheniramine maleate. Preferably, the amount present in the day time formulation is from about 0.04 to about 0.06 percent.

In accordance with an embodiment of the present invention, medicant comprises a formulation that reduces or relieves pain. In a preferable embodiment, the pain reliever is an analgesic. In a more preferable embodiment, the analgesic is acetaminophen. Preferably, the analgesic is present in an amount from about 9 to about 11 percent.

Generally speaking, in accordance with various embodiments of the present invention, a solvent may be used to dissolve active ingredients into solution. For example, in accordance with various embodiments of the present invention, propylene glycol and polyethylene glycol is present in an amount suitable to sufficiently dissolve all the active ingredients. For example, in embodiments of the present invention such as those desired above, the propylene glycol may be present in an amount from about 35 to about 50 percent and the polyethylene glycol is present in an amount from about 15 to about 25 percent.

As noted above, medicant preferably has a viscosity sufficient to remain stable and contained in the delivery portion until administered and with little or no spillage when handled. In this regard, high viscosity of the medicant may be achieved through the use of a thickening solution which is soluble in any drinkable liquid at any temperature. Generally, thickening solutions capable of achieving high viscosity while being in solution with the plurality of active ingredients are suitable. Preferably, the thickening solution comprises glycerin, hydroxyethylcellulose, and carboxymethylcellulose. In accordance with an embodiment of the present invention, the thickening solution is present in an amount from about 3 to about 5 percent, preferably from about 4 to about 4.5 percent. For example, in accordance with an embodiment of the invention, glycerin is present in an amount of about 3.4 percent; hydroxyethylcellulose is present in an amount of about 0.085 percent; and carboxymethylcellulose is present in an amount of about 0.935 percent.

In accordance with various embodiments of the present invention, various pH-adjusting agents known or as yet unknown in the art and can be used to bring the pH of the composition of the present invention to within a preferred range. For example, in accordance with one embodiment, the pH of the medicant is adjusted to be from about 3 to about 8. In a preferred embodiment, the pH of the medicant is from about 3.5 to about 7. For example, in one embodiment of the present invention, the pH-adjusting agent is citric acid and/or trisodium citrate dihydrate. In this embodiment of the invention, citric acid and trisodium citrate dihydrate are present in an amount effective to adjust the pH of the composition to within the preferred pH range, generally in an amount from about 0.32 percent to about 0.34 percent and from 0.5 to about 0.6 percent, respectively.

The following is a non-limiting example of one embodiment of the invention.

| Component | % |
|---|---|
| Glycerin USP 99.7% | 3.4 |
| HEC 250 HX | 0.085 |
| CMC 12M31XP | 0.935 |
| Citric Acid | 0.338 |
| Trisodium Citrate Dihydrate | 0.522 |
| Propylene Glycol USP | 47.059 |
| Polyethlyene Glycol 400 (PEG-8) | 15.676 |
| Dextromethorphan HBr | 0.420 |
| Phenylephrine HCl | 0.140 |
| Doxylamine Succinate | 0.175 |
| Splenda Sucralose Powder | 0.400 |
| Acetaminophen | 9.100 |
| Color Solution | 0.750 |
| Water | balance |

In accordance with various embodiments of the present invention, medicant may comprise additional optional ingredients to create suitable aesthetics or similar properties. For example, in some embodiments, a color solution may be added to create an appealing appearance. In accordance with another exemplary embodiment of the present invention, the medicant may include a flavoring agent. In a preferable embodiment of the present invention, the medicant may be flavor-neutral so that it does not interfere with the taste of the beverage into which the medicant is mixed.

Finally, it should be understood that various principles of the invention have been described in illustrative embodiments. However, many combinations and modifications of the above-described structures, arrangements, proportions, elements, materials and components, used in the practice of the invention, in addition to those not specifically described, may be varied and particularly adapted to specific environments and operating requirements without departing from those principles. Other variations and modifications of the present invention will be apparent to those of ordinary skill in the art, and it is the intent of the appended claims that such variations and modifications be covered.

1. A medicant delivery system comprising:
a delivery device including a delivery portion and a handle portion; and
a medicant having a viscosity of between about 6,000 to about 20,000 centipoise.

2. The medicant delivery device of claim 1, wherein said delivery portion and said handle portion form a utensil.

3. The medicant delivery device of claim 2, wherein said utensil is a spoon.

4. The medicant delivery device of claim 1, wherein said medicant comprises:
a plurality of active ingredients selected from the group of a cough suppressant, a decongestant, an antihistamine, and an analgesic;
a thickener; and
a pH adjustment solution wherein pH of said medicant is about 3 to about 8.

5. The medicant of claim 4, wherein said thickener comprises glycerin, hydroxyethylcellulose, and carboxymethylcellulose.

6. A high viscosity multi-symptom relieving medicant comprising:
a plurality of active ingredients selected from the group of a cough suppressant, a decongestant, an antihistamine, an expectorant and an analgesic;
a thickener wherein the viscosity of the medicant is about 6,000 to about 20,000 centipoise; and
a pH adjustment solution wherein pH is about 3 to about 8.

7. The medicant of claim 22, wherein said cough suppressant is present in solution from about 0.3 to about 0.5 percent.

8. The medicant of claim 7, wherein said cough suppressant is dextromethorphan HBr.

9. The medicant of claim 6, wherein said decongestant contains pseudoephedrine.

10. The medicant of claim 22, wherein said decongestant is present in solution from about 0.1 to about 0.15 percent.

11. The medicant of claim 10, wherein said decongestant is phenylephrine HCl.

12. The medicant of claim 22, wherein said antihistamine is present in solution from about 0.03 to about 0.18 percent.

13. The medicant of claim 12, wherein said antihistamine is doxylamine succinate or chlorpheniramine maleate.

14. The medicant of claim 6, wherein said analgesic contains an NSAID.

15. The medicant of claim 22, wherein said analgesic is present in solution from about 9 to about 11 percent.

16. The medicant of claim 15, wherein said analgesic is acetaminophen.

17. The medicant of claim 22, wherein said thickener is present in solution from about 3 to about 5 percent.

18. The medicant of claim 17, wherein said thickener comprises glycerin, hydroxyethylcellulose, and carboxymethylcellulose.

19. The medicant of claim 6, wherein said pH adjustment solution is selected from the group of citric acid anhydrous, trisodium citrate dihydrate and mixtures thereof.

20. The medicant of claim 6, further comprising propylene glycol and polyethylene glycol.

21. The medicant of claim 22, wherein said propylene glycol and polyethylene glycol is present in solution from about 60 to about 65 percent.

22. The medicant of claim 6, wherein said medicant is configured as a single dose of 5-8 grams.

23. The medicant of claim 20, wherein said propylene glycol and polyethylene glycol is present in solution from about 15 to about 75 percent.

24. A high viscosity multi-symptom relieving medicant comprising:
at least one of an active ingredient selected from the group of a cough suppressant, a decongestant, an antihistamine, an expectorant, and an analgesic;
a thickener wherein the viscosity of the medicant is about 6,000 to about 20,000 centipoise; and
a pH adjustment solution wherein pH is about 3 to about 8.
